(19) United States
(12) Patent Application Publication
(10) Pub. No.: US 2021/0404960 A1
(43) Pub. Date: Dec. 30, 2021

(54) DETECTION METHOD AND DETECTION DEVICE

(71) Applicant: Panasonic Corporation, Osaka (JP)

(72) Inventors: TATSUROU KAWAMURA, Kyoto (JP); HIROTO YANAGAWA, Osaka (JP); MASATO YASUURA, Ibaraki (JP); MAKOTO FUJIMAKI, Ibaraki (JP)

(21) Appl. No.: 17/472,794

(22) Filed: Sep. 13, 2021

Related U.S. Application Data
(63) Continuation of application No. PCT/JP2020/017894, filed on Apr. 27, 2020.

(30) Foreign Application Priority Data
May 28, 2019 (JP) 2019-099495

(51) Int. Cl. G01N 21/64; G01N 21/552; G01N 33/553

(52) U.S. Cl. CPC G01N 21/648; G01N 33/553; G01N 21/554

(57) ABSTRACT

A detection method including forming a complex by binding, to a target substance, a first substance immobilized to a metal particle with magnetism and a second substance labeled with a fluorescent material, moving the complex by applying a magnetic field, illuminating the complex during movement with excitation light of a predetermined wavelength, the excitation light causing the fluorescent material to emit fluorescence, the fluorescence being enhanced by localized surface plasmon resonance that is produced by the metal particle, capturing the enhanced fluorescence over time and obtaining two-dimensional images, and detecting the target substance in accordance with a light spot included in each of the two-dimensional images, the metal particle including an inner core made of a magnetic material and an outer shell covering the inner core, the outer shell being made of a nonmagnetic metal material that produces the localized surface plasmon resonance.

### BACKGROUND

### 1. Technical Field

The present disclosure relates to a detection method and a detection device for optically detecting a target substance present in a liquid. More particularly, the present disclosure relates to a detection method and a detection device each utilizing a surface enhanced fluorescence spectroscopy with which fluorescence is enhanced by the action of localized surface plasmon resonance that is produced by metal nanoparticles.

### 2. Description of the Related Art

Recently, because of problems such as the spread of infections caused by pathogens and the emergence of new pathogens, development of a device capable of detecting those pathogens has been urgently needed. As targets to be detected (namely, detection substances), there are known molecules such as pathogenic proteins, viruses (such as capsid proteins), and bacteria (such as polysaccharides). A highly sensitive sensor utilizing the surface enhanced fluorescence spectroscopy to detect those target substances has been disclosed (see, for example, Japanese Unexamined Patent Application Publication No. 2008-216046).

Japanese Unexamined Patent Application Publication No. 2008-216046 uses a detection antibody in which a metal particulate and a fluorescent material are integrated with each other. The detection antibody enables a trace amount of the target substance to be detected because fluorescence emitted from the fluorescent material is enhanced by the plasmon resonance that is produced by the metal particulate.

### SUMMARY

With the above-described sensor disclosed in Japanese Unexamined Patent Application Publication No. 2008-216046, however, fluorescence emitted from the detection antibody in a free state not bound to the target substance is also enhanced by the plasmon resonance. In other words, background light increases and hence detection sensitivity decreases. A method of increasing the detection sensitivity by removing the detection antibody in the free state is one conceivable solution. However, such a method needs complicated operations and takes a longer time for the detection.

One non-limiting and exemplary embodiment provides a detection method capable of increasing sensitivity in detection of a target substance with the surface enhanced fluorescence spectroscopy.

In one general aspect, the techniques disclosed here feature a detection method including forming a complex by binding, to a target substance, a first substance immobilized to a metal particle with magnetism and a second substance labeled with a fluorescent material, moving the complex by applying a magnetic field, illuminating the complex during movement with excitation light of a predetermined wavelength, the excitation light causing the fluorescent material to emit fluorescence, the fluorescence being enhanced by localized surface plasmon resonance that is produced by the metal particle, capturing the enhanced fluorescence over time and obtaining two-dimensional images, and detecting the target substance in accordance with a light spot included in each of the two-dimensional images, the metal particle including an inner core made of a magnetic material and an outer shell covering the inner core, the outer shell being made of a nonmagnetic metal material that produces the localized surface plasmon resonance.

It should be noted that general or specific embodiments of the present disclosure may be each implemented as a system, a device, an integrated circuit, a computer program, or a computer-readable recording medium, or any selective combination of two or more among the device, the system, the method, the integrated circuit, the computer program, and the recording medium. The computer-readable recording medium includes, for example, a nonvolatile recording medium such as CD-ROM (compact disk-read only memory).

The detection method according to the embodiment of the present disclosure can realize an increase of sensitivity in detection of the target substance with the surface enhanced fluorescence spectroscopy. Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 illustrates a structure of a complex in a first embodiment;

FIG. 2 illustrates a structure of another complex in the first embodiment;

FIG. 3 is a sectional view of a metal particle in the first embodiment;

FIG. 4 is an explanatory view of an enhancement phenomenon in the first embodiment;

FIG. 5 is an explanatory view of another enhancement phenomenon in the first embodiment;

FIG. 6 illustrates a configuration of a detection device according to the first embodiment;

FIG. 7 illustrates an example of a two-dimensional image obtained by the detection device according to the first embodiment;

FIG. 8 is a flowchart illustrating processing executed by the detection device according to the first embodiment;

FIG. 9 illustrates a configuration of a detection device according to a second embodiment;

FIG. 10 is a flowchart illustrating processing executed by the detection device according to the second embodiment;

FIGS. 11A and 11B each illustrate a positional relationship between the metal particle and a fluorescent material in EXAMPLE 1;

FIG. 12 is an explanatory view of a simulation model in EXAMPLES 1 and 2;

FIG. 13 is a graph depicting wavelength dependency of intensity of an electric field near the metal particle in EXAMPLE 1;

FIG. 14 is a graph depicting an extinction spectrum and a fluorescence spectrum of the fluorescent material in EXAMPLE 1;

FIGS. 15A and 15B each illustrate a positional relationship between the metal particle and the fluorescent material in EXAMPLE 2;

FIG. 16 is a graph depicting wavelength dependency of intensity of an electric field near the metal particle in EXAMPLE 2; and

FIG. 17 is a graph depicting distance dependency of the intensity of the electric field near the metal particle in EXAMPLES 1 and 2.

### DETAILED DESCRIPTION

### Underlying Knowledge Forming Basis of the Present Disclosure

A method utilizing fluorescence (hereinafter called a “fluorescence method”) is widely known as a technique for detecting molecules, viruses, and bacteria, such as proteins present in a liquid. According to the fluorescence method, a target substance and an antibody labeled with a fluorescent material (hereinafter called a “fluorescent labeled antibody”) are bound to each other by an antigen-antibody reaction. Then, the fluorescent labeled antibody bound to the target substance is illuminated with light capable of exciting the fluorescent material, thus causing the fluorescent material to generate fluorescence. The target substance can be detected by detecting the fluorescence generated as described above.

### Fluorescence Polarization Immunoassay

Fluorescence polarization immunoassay is known as an example of the fluorescence method. According to the fluorescence polarization immunoassay, a test solution containing an antigen, namely a target substance, is mixed with a solution containing the fluorescent labeled antibody. As a result, a complex is formed by the antigen-antibody reaction. At that timing, a concentration of the antigen is measured based on a difference in polarization degree of fluorescence between before and after the formation of the complex. This immunoassay utilizes a phenomenon that the polarization degree is increased with the formation of the complex because the complex has a greater size than a single fluorescent labeled antibody and suppresses rotational motion (see, for example, Japanese Unexamined Patent Application Publication No. 62-38363 and Michael E. Jolley et al. Clinical Chemistry, vol. 27, No7(1981)).

### Immunochromatography

Immunochromatography is known as another example of the fluorescence method. This method uses a flat substrate including a base formed of, for example, a nitrocellulose membrane. An antibody capable of specifically binding to a target substance is immobilized onto the substrate. When a sample solution containing the target substance and the fluorescent labeled antibody is dropped onto the substrate, the target substance bound to the fluorescent labeled antibody is captured by the antibody immobilized onto the substrate (hereinafter called the “immobilized antibody”). By illuminating the captured antibody with light, fluorescence is emitted at intensity corresponding to a concentration of the target substance. The concentration of the target substance can be measured by detecting the emitted fluorescence (see, for example, Japanese Unexamined Patent Application Publication No, 2001-133455).

### Utilization of Surface Enhanced Fluorescence in Immunochromatography

There is also known a technique of utilizing surface enhanced fluorescence to increase sensitivity of the Immunochromatography. According to this method, a region including a metal nanostructure is formed in a flow path through which a solution flows, and an antibody capable of binding to a target substance is immobilized onto the metal nanostructure. When a sample solution containing the target substance and the fluorescent labeled antibody is dropped into the flow path, the target substance bound to the fluorescent labeled antibody is captured by the immobilized antibody. By illuminating the metal nanostructure with light of a wavelength at which the localized surface plasmon resonance is excited, fluorescence emitted from the fluorescent labeled antibody is enhanced. Intensity of the fluorescence enhanced by the localized surface plasmon resonance (hereinafter also called “surface enhanced fluorescence”) increases depending on a concentration of the target substance. Because a degree at which the fluorescence is enhanced (hereinafter called an “enhancement degree”) is about 10 to 1000 times, the surface enhanced fluorescence has about 10 to 1000 times higher intensity than ordinary fluorescence. Hence the target substance of such a low concentration as not being measured with use of the ordinary fluorescence can be measured by utilizing the surface enhanced fluorescence (see, for example, Japanese Unexamined Patent Application Publication No. 2010-19765).

Utilization of Evanescent Wave in Immunochromatography

There is further known a method using a backside illumination system that illuminates a transparent substrate with excitation light from the backside. According to this method, a rear surface of the substrate is illuminated with the excitation light, and an evanescent wave is induced. The induced evanescent wave is applied to the fluorescent labeled antibody that is captured by the immobilized antibody on a front surface of the substrate, thus causing the fluorescent labeled antibody to emit fluorescence. At that time, because the evanescent wave is applied to a region at several hundred nm from the front surface of the substrate, an amount of the excitation light applied to the fluorescent labeled antibody not bound to the target substance (hereinafter called a “Free component”) can be reduced in comparison with the case of illuminating the front side of the substrate. The fluorescence emitted from the Free component is a noise component not reflecting the concentration of the target substance. In other words, the fluorescence emitted from the Free component impedes measurement of the fluorescence emitted from the fluorescent labeled antibody bound to the target substance and reduces the measurement accuracy. It is hence effective to limit an illumination region and to suppress the fluorescence emitted from the Free component by utilizing the evanescent wave (see, for example, Japanese Unexamined Patent Application Publication No. 2008-216046).

### Flowcytometry

As a detection method for a particle and a target substance, there is a method called flowcytometry. According to the flowcytometry, particles, such as cells, flowing through a transparent thin tube (flow cell) one by one is illuminated with a laser beam, for example, upon which scattered light and/or fluorescence light is generated. The particles are identified and counted by detecting the scattered light and/or the fluorescence light generated as mentioned above.

When detecting the target substance such as a protein, a detection process is performed as follows. First; two types of antibodies capable of specifically binding to the target substance are prepared. One of the antibodies is immobilized to a capturing bead, and the other antibody is labelled with a fluorescent material. Those two types of antibodies are each caused to develop an antigen-antibody reaction with the target substance to form specific binding (sandwich binding) with the target substance sandwiched between the two types of antibodies, thus forming a complex of the capturing bead—the target substance—the fluorescent labeled antibody. After removing the unbound fluorescent labeled antibody from a solution containing the complex, the solution is supplied to flow through the flow cell. At that timing, the fluorescence generated from the complex is detected, and the target substance is identified and counted (see, for example, Kathryn L. Kellar et al. Experimental Hematology 301227-1237(2002) and AimPlex_Multiplex_Immunoassay_User_Manual Rev 1.3.24).

### EFA-NI (External Force-Assisted Near-Field Illumination) Biosensor

There is further known a method called an external force-assisted near-field illumination (EFA-NI) biosensor capable of counting target substances, such as particles, for example, viruses, and proteins, one by one. According to this method, two types of antibodies capable of specifically binding to the particle or the target substance are first prepared. One of the antibodies is immobilized to a magnetic particle with magnetism, and the other antibody is labelled with a fluorescent material or a fluorescent microparticle. Those two types of antibodies are mixed with a test solution containing the particle or the target substance to prepare a mixed solution. An antigen-antibody reaction progresses in the mixed solution, and the two types of antibodies cause specific binding (sandwich binding) with the particle or the target substance sandwiched between the two types of antibodies, thus forming an antigen-antibody complex. The mixed solution is held on a front surface of a detection plate that produces a near field in the front surface when the detection plate is illuminated with light from the backside. A magnetic field is applied perpendicularly to the detection plate, whereby the antigen-antibody complex in the mixed solution is attracted toward near the front surface of the detection plate. At that timing, the antigen-antibody complex is illuminated with the near field and a two-dimensional image of the mixed solution is captured from the front side of the detection plate. The antigen-antibody complex appears as a light spot emitting fluorescence on the two-dimensional image. Then, when a magnetic field is applied parallel to the front surface of the detection plate, the light spot on the two-dimensional image is moved in a direction of the magnetic field. The number of the particles or the target substances can be measured one by one by counting the light spot that is moved as mentioned above (namely, the moving light spot) (see, for example, International Publication No. 2017/187744 and Yasuura, M. and Fujimaki, M. Sci. Rep. 6, 39241; doi: 10.1038/srep39241 (2016)).

However, the above-described methods have the following problems.

With the fluorescence polarization immunoassay, because of measuring the difference in the polarization degree, a polarizer rotation mechanism is required, and a device is complicated. Furthermore, the difference in the polarization degree reflects a difference in size between before and after the formation of the antigen-antibody complex. Therefore, when a molecule smaller than the fluorescent labeled antibody is to be detected, the difference in the polarization degree is small and hence the detection accuracy decreases. Another problem is, for example, that, if a scattering substance is present in the test solution, the difference in the polarization degree cannot be detected due to depolarization.

With each of the immunochromatography and the method utilizing the surface enhanced fluorescence or the evanescent wave, there is a possibility that the fluorescent labeled antibody not bound to the target substance or a coexisting substance emitting fluorescence may be nonspecifically adsorbed to the substrate. This gives rise to a problem that the detection accuracy may decrease with the fluorescence emitted from both the fluorescent labeled antibody and the coexisting substance which have been nonspecifically adsorbed to the substrate.

The flowcytometry needs a step of removing the fluorescent labeled antibody not bound to the target substance and takes a longer measurement time.

With the EFA-NI biosensor, the fluorescent labeled antibody not bound to the target substance is also illuminated with the near field and generates fluorescence. Therefore, brightness of a background image (namely, a background level) rises, and the light spot becomes difficult to recognize. Accordingly, the number of the fluorescent labeled antibodies not bound to the target substance needs to be reduced, and the number of the fluorescent labeled antibodies mixed with the test solution has to be limited.

Because the number of the target substances capable of being quantitated is smaller than that of the fluorescent labeled antibodies, a quantitative range is limited as a result of limiting the number of the fluorescent labeled antibodies to be mixed with the test solution.

### SUMMARY OF PRESENT DISCLOSURE

A detection method according to one aspect of the present disclosure includes forming a complex by binding, to a target substance, a first substance immobilized to a metal particle with magnetism and a second substance labeled with a fluorescent material, moving the complex by applying a magnetic field, illuminating the complex during movement with excitation light of a predetermined wavelength, the excitation light causing the fluorescent material to emit fluorescence, the fluorescence being enhanced by localized surface plasmon resonance that is produced by the metal particle, capturing the enhanced fluorescence over time and obtaining two-dimensional images, and detecting the target substance in accordance with a light spot included in each of the two-dimensional images, the metal particle including an inner core made of a magnetic material and an outer shell covering the inner core, the outer shell being made of a nonmagnetic metal material that produces the localized surface plasmon resonance.

With the features described above, the fluorescence emitted from the fluorescent material labeling the second substance contained in the complex is enhanced by the action of the localized surface plasmon resonance that is produced by the metal particle immobilized to the first substance contained in the complex. On the other hand, the fluorescent material labeling the second substance not contained in the complex (namely, the second substance in a free state) is not positioned spatially close to the metal particle, and hence the fluorescence emitted from that fluorescent material is hardly enhanced by the localized surface plasmon resonance. Accordingly, the fluorescence emitted from the fluorescent material labeling the second substance contained in the complex appears as a light spot in each of the two-dimensional images with higher brightness than a light spot formed by the fluorescence emitted from the fluorescent material labeling the second substance not contained in the complex. Hence the complex can be more easily detected on the two-dimensional image with no need of removing the second substance in the free state, and sensitivity in detection of the target substance can be increased with the measurement using a higher-speed and simpler surface enhanced fluorescence spectroscopy.

Furthermore, since the target substance can be detected without using polarized light, a device configuration can be simplified. Moreover, an influence due to a difference in molecule size between before and after the formation of the complex can be reduced, and an application range of the target substance can be widened.

The target substance can be detected in accordance with the light spot moving over the two-dimensional images, and influences of contaminations being not moved by the magnetic field can be reduced. In addition, since the first substance emits no fluorescence, the first substance not contained in the complex can be suppressed from being falsely detected as the target substance. Thus, since the target substance can be detected even when the sample contains large amounts of the first substance and the second substance in the free state, concentrations of the first substance and the second substance in the sample can be increased. As a result, it is possible to increase an amount of the target substance capable of forming the complex and to expand a concentration range in which the target substance can be quantitated.

Since the fluorescence emitted from the complex is enhanced, a sharper two-dimensional image is obtained. Accordingly, image recognition of the moving light spot is facilitated, and false recognition can be reduced. Furthermore, since the fluorescence emitted from the complex is enhanced, a fluorescent material with a smaller diameter size can be utilized. As a result, a formation speed of the complex can be increased, and speedup of the detection can be realized.

Since, in each of the metal particles, the inner core made of the magnetic material can be covered with the outer shell made of the nonmagnetic metal material, the metal particles can be suppressed from agglomerating together due to residual magnetization of the magnetic material. As a result, it is possible to suppress variations in brightness and moving speed of the light spot among the two-dimensional images and to increase the detection accuracy.

Moreover, since a surface of the metal particle can be covered with the metal material that produces the localized surface plasmon resonance, a variation in enhancement degree in a circumferential direction of the metal particle can be suppressed in comparison with the case in which part of the surface of the metal particle is made of the metal material.

For example, in the detection method according to the one aspect of the present disclosure, the illuminating the complex with the excitation light may include illuminating a substrate capable of forming a near field induced by the excitation light with the excitation light, thereby the complex positioned near a surface of the substrate being illuminated with the near field induced by the excitation light.

With the feature described above, the fluorescent material present near the surface of the substrate can be selectively illuminated with the near field, and emission of fluorescence from the fluorescent material positioned away from the surface of the substrate can be suppressed. Accordingly, an influence upon the detection by the fluorescent material located in a region where the near field is not illuminated can be reduced.

For example, in the detection method according to the one aspect of the present disclosure, the applying the magnetic field may include applying a first magnetic field, thereby the complex being attracted toward the surface of the substrate, and applying a second magnetic field, thereby the complex attracted to the surface of the substrate moving along the surface of the substrate.

With the feature described above, since the complex can be attracted to the surface of the substrate and then moved along the surface of the substrate, the fluorescent material contained in the complex can be effectively illuminated with the near field induced by the excitation light. On the other hand, since the fluorescent material not contained in the complex is not attracted toward the surface of the substrate; the fluorescent material not contained in the complex can be suppressed from being illuminated with the near field induced by the excitation light. Accordingly, in the two-dimensional images, the influence of the fluorescence generated from the fluorescent material not contained in the complex of the target substance can be reduced, and the detection accuracy of the target substance can be further increased.

For example, in the detection method according to the one aspect of the present disclosure, the magnetic material may contain a paramagnetic substance, and the nonmagnetic metal material may contain a diamagnetic substance.

With the features described above, the metal particles can be suppressed from agglomerating together when the magnetic field is not applied.

For example, in the detection method according to the one aspect of the present disclosure, the nonmagnetic metal material may be gold, silver, aluminum, or an alloy containing, as a main component, one of gold, silver, and aluminum.

With the feature described above, since gold, silver, aluminum, or the alloy containing one of those metals as a main component is used to form the outer shell, the localized surface plasmon resonance can be effectively produced by the metal particle. Furthermore, when the outer shell is made of gold, coatings with various functions can be optionally easily formed on the surface of the metal particle. For example, in the case of forming a nonspecific adsorption preventive coating on the outer shell, nonspecific adsorption that the second substance labeled with the fluorescent material is adsorbed onto the surface of the metal particle can be reduced, and the incidences of false-positive and false-negative detection results can be reduced.

When the outer shell is made of gold, silver, aluminum, or the alloy containing one of those metals as a main component, a quenching phenomenon tends to occur in the fluorescent material labeling the second substance that has been non-specifically adsorbed onto the surface of the metal particle. The term “quenching phenomenon” implies a fluorescence quenching phenomenon due to direct transfer of energy from the fluorescent material to the metal particle. In the case of the nonspecific adsorption, because a distance between the fluorescent material and the surface of the metal particle is reduced, fluorescence quenching caused by the above-described quenching phenomenon becomes significant. It is hence possible to suppress intensity of the fluorescence generated due to the nonspecific adsorption and to detect the target substance more accurately.

The above-described general or specific embodiments may be implemented as a system, a device, an integrated circuit, a computer program, or a computer-readable recording medium such as CD-ROM, or any selective combination of two or more among the device, the system, the method, the integrated circuit, the computer program, and the recording medium.

The embodiments will be described below with reference to the drawings.

It is to be noted that any of the following embodiments represents a general or specific example. Numerical values, shapes, materials, constituent elements, layout positions and connection forms of the constituent elements, steps, order of the steps, etc., which are described in the following embodiments, are merely illustrative, and they are not purported to limit the scope of the present disclosure. Among the constituent elements in the following embodiments, those ones not stated in independent Claims are explained as optional constituent elements. The drawings are not always exactly drawn in a strict sense. In the drawings, substantially the same constituent elements are denoted by the same reference sings, and duplicate description of those constituent elements is omitted or simplified in some cases.

In the following description, terms representing relationships between the constituent elements, such as “parallel” and “vertical”, terms representing shapes of the constituent elements, such as “cylindrical”, and a numerical range are to be interpreted as not exactly expressing the meaning in a strict sense but as including substantially equivalent meaning and range with an allowance of serval %, for example.

Furthermore, in the following description, the wording “detect the target substance” implies not only a process of finding the target substance and checking the presence of the target substance, but also a process of measuring an amount (for example, number or concentration) of the target substances or a range of the amount.

### First Embodiment

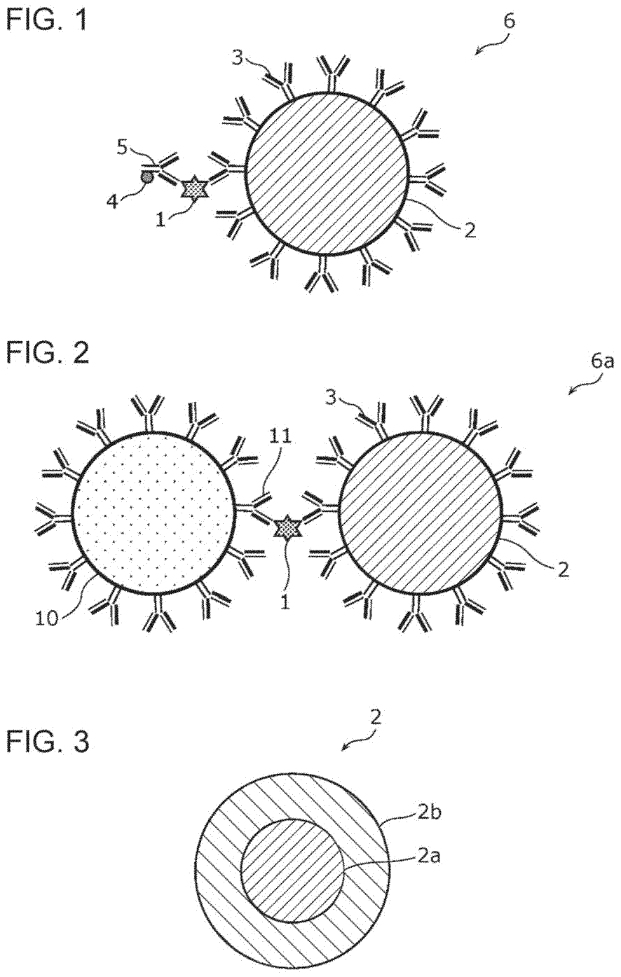

A first embodiment is described below with reference to FIGS. 1 to 8.

### Structure of Complex

First, a structure of a complex 6 is described in detail with reference to FIG. 1. FIG. 1 illustrates the structure of the complex 6 in the first embodiment. As illustrated in FIG. 1, the complex 6 includes a target substance 1, a first substance 3 immobilized to a metal particle 2, and a second substance 5 labeled with a fluorescent material 4. A known method, such as physical adsorption, covalent binding, ion binding, or cross-linking, is used as a method of immobilizing the first substance 3 to the metal particle 2. A method of labeling the second substance 5 with the fluorescent material 4 may be a method of binding the second substance 5 and the fluorescent material 4 with physical adsorption, covalent binding, ion binding, or cross-linking.

The target substance 1 is a molecule to be detected, for example, a protein or the like.

The metal particle 2 has paramagnetism or ferromagnetism and produces localized surface plasmon resonance when illuminated with excitation light of a predetermined wavelength. An internal structure of the metal particle 2 will be described later with reference to FIG. 3.

The first substance 3 is an antibody capable of specifically binding to the target substance 1. The first substance 3 is immobilized to a surface of the metal particle 2. Although first substances 3 are immobilized to the metal particle 2 in FIG. 1, the present disclosure is not limited to such a case. For example, the number of the first substances 3 immobilized to the metal particle 2 may be one.

The fluorescent material 4 emits fluorescence when illuminated with excitation light of a predetermined wavelength. The fluorescent material 4 is made of, for example, an organic molecule or a quantum dot.

The second substance 5 is an antibody capable of specifically binding to the target substance 1 and is labeled with the fluorescent material 4, In other words, the second substance 5 is the fluorescent labeled antibody. Although the second substance 5 is labeled with one fluorescent material 4 in FIG. 1, the second substance 5 may be labeled with fluorescent materials.

Here, the first substance 3 and the second substance 5 are different. The wording “the first substance 3 and the second substance 5 are different” implies that any parts of the first substance 3 immobilized to the fluorescent material 4 and the second substance 5 immobilized to the metal particle 2 are not shared, and that the first substance 3 and the second substance 5 are present as separate substances. Each of the first substance 3 and the second substance 5 is just required to have properties of specifically binding to the target substance 1, and molecular structures of both the substances are not limited to particular ones. The first substance 3 and the second substance 5 may be different types of molecules or similar types of molecules.

The first substance 3 and the second substance 5 are bound to different sites of the target substance 1. Accordingly, as illustrated in FIG. 1, the first substance 3 and the second substance 5 bind to each other with the target substance 1 sandwiched therebetween (called sandwich binding), thus forming the complex 6.

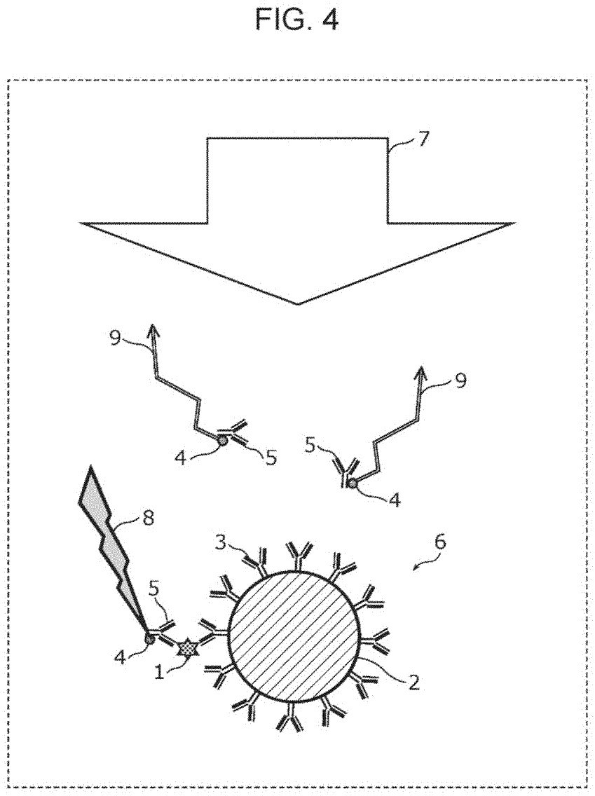

A structure of another complex 6a will be described below with reference to FIG. 2. FIG. 2 illustrates a structure of the complex 6a in the first embodiment.

In the complex 6a, as illustrated in FIG. 2, a fluorescent particle 10 is used instead of the fluorescent material 4. In other words, the complex 6a includes the target substance 1, the first substance 3 immobilized to the metal particle 2, and a second substance 11 labeled with the fluorescent particle 10.

The fluorescent particle 10 made of resin (for example, polystyrene or acryl) or glass into which, for example, an organic fluorescent molecule, an inorganic fluorescent molecule, or a quantum dot is incorporated. The fluorescent particle 10 has a diameter of several ten nm to several hundred nm.

The fluorescent particle 10 can be given with characteristics that are difficult to realize with the fluorescent material 4 alone. For example, light fading of the fluorescent particle 10 can be reduced by adding a deactivation inhibitor for the fluorescence to the resin or the glass forming the fluorescent particle 10. Furthermore, various surface modifications using an amino group or a carboxyl group, for example, can be made on the fluorescent particle 10. Moreover, the fluorescent particle 10 can provide higher dispersibility in water than the fluorescent material 4. In addition, since the fluorescent particle 10 has a greater size than the fluorescent material 4, it is relatively easy to observe even the single fluorescent particle 10.

The second substance 11 is an antibody capable of specifically binding to the target substance 1 and is labeled with the fluorescent particle 10. In other words, the second substance 11 is the fluorescent labeled antibody. As in the case of FIG. 1, the first substance 3 and the second substance 11 are bound to different sites of the target substance 1. Accordingly, as illustrated in FIG. 2, the first substance 3 and the second substance 11 bind to each other with the target substance 1 sandwiched therebetween (called sandwich binding), thus forming the complex 6a.

### Internal Structure of Metal Particle

The internal structure of the metal particle 2 is described below with reference to FIG. 3. FIG. 3 is a sectional view of the metal particle 2 in the first embodiment. As illustrated in FIG. 3, the metal particle 2 includes an inner core 2a and an outer shell 2b.

The inner core 2a is made of a magnetic material with paramagnetism or ferromagnetism. The term “paramagnetism” implies magnetism that does not cause magnetization in the absence of an external magnetic field, and that, upon application of a magnetic field, causes weak magnetization in a direction of the applied magnetic field. The term “ferromagnetism” implies magnetism that can cause spontaneous magnetization even in the absence of an external magnetic field. Thus, the inner core 2a is moved in the direction of the magnetic field with the application of the magnetic field.

In this embodiment, a magnetic material with the paramagnetism is used. More specifically, ferrite containing iron oxide as a main raw material is used. The magnetic material is not limited to the ferrite containing iron oxide as the main raw material. For example, iron may be used as the magnetic material.

The outer shell 2b covers the inner core 2a and is made of a nonmagnetic metal material that produces the localized surface plasmon resonance. For example, gold, silver, or aluminum can be used as the metal material. In another example, an alloy containing, as a main component, any of gold, silver, and aluminum can also be used as the metal material.

In this embodiment, the nonmagnetic metal material includes a diamagnetic substance. Accordingly, the wording “nonmagnetic” is used here although gold and silver are called diamagnetic substances in some cases.

The metal particle 2 has a diameter of about several nm to several hundred nm. The localized surface plasmon resonance can be produced by illuminating the metal particle 2 in such a diameter range with light of a predetermined wavelength. When a wavelength range in which the localized surface plasmon resonance is produced overlaps with a wavelength range of the light exciting the fluorescent material 4 and/or a wavelength of the fluorescence emitted from the fluorescent material 4, the fluorescence emitted from the fluorescent material 4 present near the metal particle 2 is enhanced by the action of the localized surface plasmon resonance. That enhanced fluorescence is called “surface enhanced fluorescence”

Enhancement Phenomenon of Fluorescence with Localized Surface Plasmon Resonance

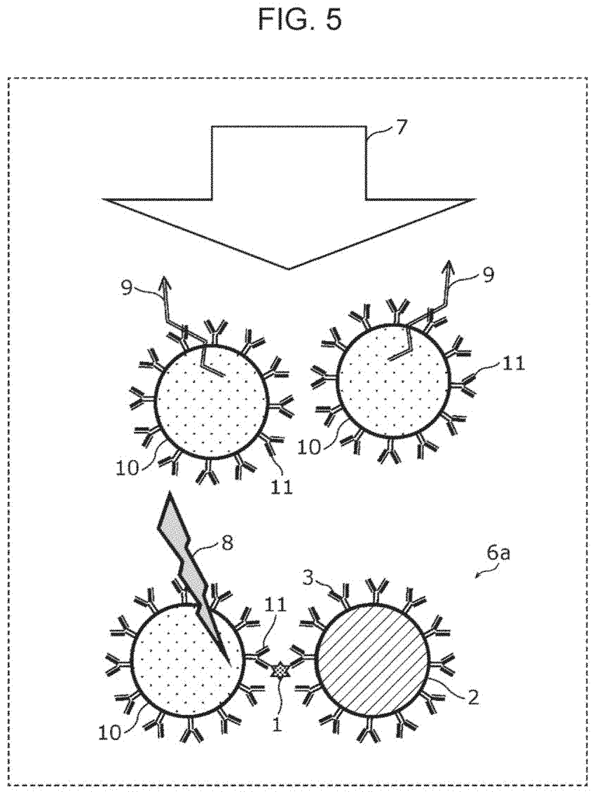

A detection device according to this embodiment utilizes an enhancement phenomenon with the localized surface plasmon resonance. The enhancement phenomenon is summarized with reference to FIGS. 4 and 5.

FIGS. 4 and 5 are explanatory views of the enhancement phenomenon in the first embodiment.

FIG. 4 represents a mixed solution (namely, a sample) containing the complex 6 and the second substance 5 both illustrated in FIG. 1. When this mixed solution is illuminated with excitation light 7 of a predetermined wavelength, the localized surface plasmon resonance is produced by the metal particle 2, and fluorescence is emitted from the fluorescent material 4.

On that occasion, the fluorescence emitted from the fluorescent material 4 bound to the second substance 5 contained in the complex 6 is enhanced by the action of the localized surface plasmon resonance produced by the metal particle 2 and is emitted as surface enhanced fluorescence 8.

On the other hand, the fluorescent material 4 bound to the second substance 5 not contained in the complex 6 and being in the free state is positioned away from the metal particle 2 and cannot receive the action of the localized surface plasmon resonance. Accordingly, the fluorescence emitted from that fluorescent material 4 is not enhanced and is emitted as ordinary fluorescence 9.

FIG. 5 represents a mixed solution containing the complex 6a and the second substance 11 both illustrated in FIG. 2. When this mixed solution is illuminated with the excitation light 7 of the predetermined wavelength, the localized surface plasmon resonance is produced by the metal particle 2, and fluorescence is emitted from the fluorescent particle 10.

On that occasion, the fluorescence emitted from the fluorescent particle 10 bound to the second substance 11 contained in the complex 6a is enhanced by the action of the localized surface plasmon resonance produced by the metal particle 2 and is emitted as the surface enhanced fluorescence 8.

On the other hand, the fluorescent particle 10 bound to the second substance 11 not contained in the complex 6a and being in the free state is positioned away from the metal particle 2 and cannot receive the action of the localized surface plasmon resonance. Accordingly, the fluorescence emitted from that fluorescent particle 10 is not enhanced and is emitted as the ordinary fluorescence 9.

### Configuration of Detection Device

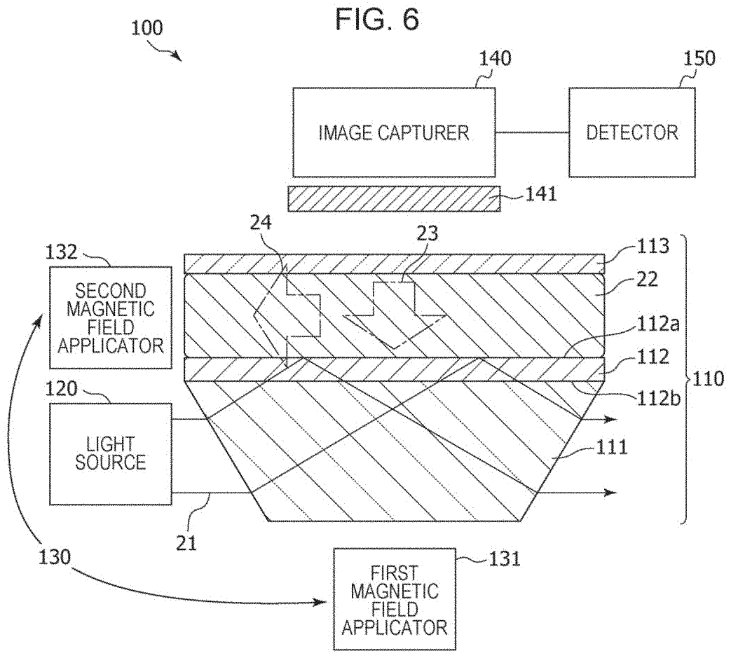

A configuration of a detection device 100 detecting the complex 6a (namely, the target substance 1) based on the above-described enhancement phenomenon will be described below with reference to FIG. 6.

FIG. 6 illustrates the configuration of the detection device 100 according to the first embodiment. The detection device 100 includes a sample container 110, a light source 120, a first magnetic field applicator 131, a second magnetic field applicator 132, an image capturer 140, a long-pass filter 141, and a detector 150. Those individual components of the detection device 100 will be described in order below.

The sample container 110 is a member in the form of a container with a space capable of containing a liquid sample. The sample container 110 contains a mixed solution 22 containing the complex 6a, the first substance 3 immobilized to the metal particle 2, and the second substance 11 labeled with the fluorescent particle 10. The sample container 110 includes a substrate 112 capable of forming a near field upon being illuminated with excitation light 21, and a prism 111. More specifically, the substrate 112 is placed on a front surface of the prism 111, and a rear surface 112b of the substrate 112 is optically bonded to the front surface of the prism 111 with the aid of refractive index adjusting oil, an optical adhesive, and so on. Thus, the substrate 112 functions as a substrate capable of forming the near field in its front surface 112a.

The term “near field” implies a thin photo-membrane produced near an object surface. For example, when light propagating from a medium with a higher refractive index into a medium with a lower refractive index and is totally reflected at an interface between those two media, the near field is formed as a very thin photo-membrane having extruded into the medium with the lower refractive index. The near field is called “near-field light” in some cases.

The sample container 110 further includes a transparent cover glass 113 covering the mixed solution 22. The mixed solution 22 is held between the substrate 112 and the cover glass 113, The sample container 110 may include a sidewall (not illustrated) surrounding the mixed solution 22. The sidewall extends from the substrate 112 toward the cover glass 113.

The light source 120 illuminates the rear surface 112b of the substrate 112 with the excitation light 21 through the prism 111. The excitation light 21 has the predetermined wavelength and is totally reflected at an interface between the mixed solution 22 and the substrate 112. As a result, the near field is formed in the front surface 112a of the substrate 112. The predetermined wavelength is set to a wavelength at which the localized surface plasmon resonance can be excited on the metal particle 2 and the fluorescence can be excited on the fluorescent particle 10. The near field is formed near the front surface 112a of the substrate 112 and sharply attenuates as a distance from the front surface 112a of the substrate 112 increases. Hence the near field induced by the excitation light 21 is applied to the mixed solution 22 near the front surface 112a of the substrate 112.

A structure of the substrate 112 is not limited to a particular one and may be selected as appropriate depending on the purpose. For example, the substrate 112 may be constituted as a single layer or a multilayer body aiming to enhance an electric field.

As illustrated in FIG. 6, the first magnetic field applicator 131 applies a first magnetic field 23 penetrating through the mixed solution 22 downward (in a direction perpendicular to the front surface 112a of the substrate 112). The first magnetic field 23 has a downward component but does not have a horizontal component. The first substance 3 immobilized to the metal particle 2 and the complex 6a in the mixed solution 22 are both attracted to the front surface 112a of the substrate 112 by the first magnetic field 23. As a result, the complex 6a and the first substance 3 present in the mixed solution 22 are illuminated with the near field induced by the excitation light 21.

As illustrated in FIG. 6, the second magnetic field applicator 132 applies a second magnetic field 24 penetrating through the mixed solution 22 horizontally (in a direction parallel to the front surface 112a of the substrate 112). The second magnetic field 24 has a horizontal component but does not have a vertical component. The first substance 3 immobilized to the metal particle 2 and the complex 6a in the mixed solution 22 are both moved along the front surface 112a of the substrate 112 by the second magnetic field 24.

For example, an electromagnet or a permanent magnet can be used as each of the first magnetic field applicator 131 and the second magnetic field applicator 132. When the electromagnet is used, each of the first magnetic field applicator 131 and the second magnetic field applicator 132 can switch over application and non-application of a magnetic field by controlling supply of a current. When the permanent magnet is used, each of the first magnetic field applicator 131 and the second magnetic field applicator 132 can switch over application and non-application of a magnetic field by moving the permanent magnet.

The image capturer 140 is constituted by, for example, an optical lens and an image sensor, and captures an image of the mixed solution 22 from the side facing the front surface 112a of the substrate 112. More specifically, the image capturer 140 captures a two-dimensional image of the surface enhanced fluorescence generated near the front surface 112a of the substrate 112 in the mixed solution 22 upon being illuminated with the excitation light 21 over time. In other words, the image capturer 140 captures the fluorescence enhanced by the localized surface plasmon resonance over time and obtains two-dimensional images. Each of the two-dimensional images includes one or more light spots.

The long-pass filter 141 cuts off the excitation light 21 and allows the fluorescence to pass therethrough. In other words, the long-pass filter 141 has a cutoff wavelength between the wavelength of the excitation light 21 and the wavelength of the fluorescence. Therefore, the image capturer 140 captures the fluorescence emitted from the vicinity of the front surface 112a of the substrate 112 but does not capture the excitation light 21. The long-pass filter 141 may be incorporated in the image capturer 140. The long-pass filter 141 is not always required to be included in the detection device 100.

The detector 150 detects the target substance 1 in accordance with one or more light spots included in each of the two-dimensional images. The detector 150 is constituted by, for example, a computer including a processor and a memory. The processor enables the target substance 1 to be detected by executing instructions or software program stored in the memory. Instead, the detector 150 may be constituted by a dedicated electronic circuit.

On each of the two-dimensional images, the fluorescent particle 10 existing near the front surface 112a of the substrate 112 and emitting the fluorescence appears as the light spot. In the complex Ga, the fluorescence emitted from the fluorescent particle 10 is enhanced by the action of the localized surface plasmon resonance produced by the metal particle 2. In addition, the metal particle 2 in the complex 6a is subjected to force from the second magnetic field 24. Accordingly, the fluorescent particle 10 contained in the complex Ga appears as a bright light spot (with high brightness) moving on each of the two-dimensional images.

On the other hand, the fluorescence emitted from the fluorescent particle 10 alone (namely, the fluorescent particle 10 not bound to the metal particle 2) is not enhanced by the localized surface plasmon resonance. Furthermore, the fluorescent particle 10 alone is not subjected to the force from the second magnetic field 24. Accordingly, when observing each of the two-dimensional images, the fluorescent particle 10 not contained in the complex 6a is recognized as a dark light spot (with low brightness) not moving on the image. The dark light spot may be buried in the background (background noise) and cannot be identified as the light spot in some cases.

The detector 150 tracks the one or more bright light spots appearing on each of the two-dimensional images and calculates a moving speed of each of the one or more light spots. Then, the detector 150 counts the light spots for which the calculated moving speed is larger than a threshold speed, and determines the number of the counted light spots to be the number of the target substances 1. As a result, the number or concentration of the target substances 1 in the mixed solution 22 can be obtained.

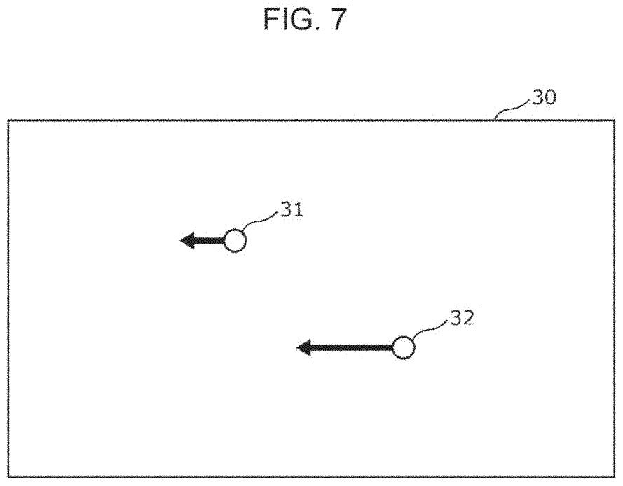

FIG. 7 illustrates an example of a two-dimensional image 30 obtained by the detection device 100 according to the first embodiment and representing a moving light spot obtained from the two-dimensional images 30. In FIG. 7, the brightness of the image including the background is reversed for easier visual recognition.

In FIG. 7, the two-dimensional image 30 includes a light spot 31 and a light spot 32 each moving in the horizontal direction. The detector 150 can count the number of the target substances 1 by counting the moving light spots 31 and 32. Although the first substance 3 alone immobilized to the metal particle 2 is also moved in the horizontal direction by the second magnetic field 24, that first substance 3 does not appear as the light spot on the two-dimensional images because it does not emit fluorescence.

### Operation of Detection Device

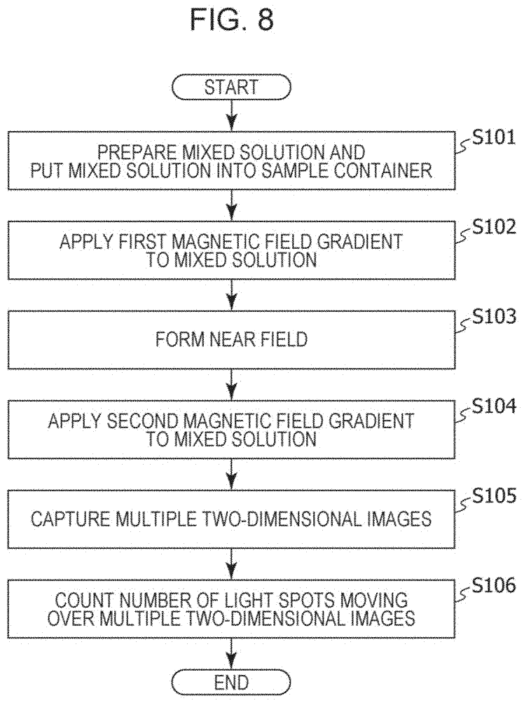

Operation of the detection device 100 with the above configuration will be described below with reference to FIG. 8. FIG. 8 is a flowchart illustrating processing executed by the detection device 100 according to the first embodiment.

First, the mixed solution 22 prepared in advance is put into the sample container 110 (S101), The mixed solution 22 containing the complex 6a is thereby placed between the substrate 112 and the cover glass 113. The mixed solution 22 is prepared by mixing a solution containing the target substance 1, a solution containing the second substance 11 labeled with the fluorescent particle 10, and a solution containing the first substance 3 immobilized to the metal particle 2 in no particular order.

The first magnetic field applicator 131 applies the first magnetic field 23 to the mixed solution 22 (S102). With the application of the first magnetic field 23, the complex 6a in the mixed solution 22 is attracted to the front surface 112a of the substrate 112. The first magnetic field 23 is applied for a predetermined period. The predetermined period is a period as long as enough for the complex 6a dispersed into the mixed solution 22 to reach the front surface 112a of the substrate 112. The length of the predetermined period is set depending on degrees of dispersity and magnetism of particles in the mixed solution 22 and the intensity of the first magnetic field 23.

Then, the light source 120 illuminates the rear surface 112b of the substrate 112 with the excitation light 21, thus forming the near field in the front surface 112a of the substrate 112 (S103). The complex 6a attracted to the front surface 112a of the substrate 112 by the first magnetic field 23 is illuminated with the near field induced by the excitation light 21.

Then, the second magnetic field applicator 132 applies the second magnetic field 24 to the mixed solution 22 (S104). With the application of the second magnetic field 24, the complex 6a in the mixed solution 22 is moved along the front surface 112a of the substrate 112. The second magnetic field 24 is applied for a period in which the illumination with the excitation light 21 is continued.

The image capturer 140 captures the fluorescence on the substrate 112 through the long-pass filter 141 over time and obtains two-dimensional images (S105). The two-dimensional images 30 obtained here are each a two-dimensional image representing the intensity of the fluorescence when viewing the front surface 112a of the substrate 112 in plan. The image capturer 140 captures the two-dimensional images at preset time intervals and obtains moving images representing change in the intensity of the fluorescence over time (namely, the two-dimensional images). The two-dimensional images are captured during a period in which the second magnetic field 24 is applied and in which the illumination with the excitation light 21 is continued.

The detector 150 analyzes the two-dimensional images and counts the light spots of which moving speeds observed on the two-dimensional images are higher than the threshold speed, thereby outputting a qualitative or quantitative detection result of the target substance 1 (S106).

### Advantageous Effects, Etc.

According to this embodiment, as described above, the light spot of which position is changed (namely, which is moved) over the two-dimensional images represents the fluorescence emitted from the complex 6a. Hence the detector 150 can detect the target substance 1 in accordance with the light spot of which position is changed over the two-dimensional images. On that occasion, the first substance 3 not bound to the target substance 1 emits no fluorescence because of not containing the fluorescent particle 10. Furthermore, the second substance 11 not bound to the target substance 1 is not moved by the first magnetic field 23 and the second magnetic field 24 because of being not associated with the metal particle 2. Thus, since the second substance 11 not bound to the target substance 1 is not attracted to the vicinity of the front surface 112a of the substrate 112, that second substance 11 is not illuminated with the near field and hardly emits the fluorescence. It is hence possible to suppress the fluorescence emitted from the first substance 3 and the second substance 11 each not bound to the target substance 1, and to suppress an increase in brightness of the background on the two-dimensional image. As a result, even when the concentrations of the first substance 3 and the second substance 11 are increased, the detection device 100 can detect the target substance 1 and can expand a quantitative range where the target substance 1 can be detected.

Furthermore, since the fluorescence emitted from the complex 6a is enhanced by the localized surface plasmon resonance, that fluorescence appears as the light spot with high brightness on the two-dimensional image. On the other hand, the fluorescence emitted from the second substance 11 not contained in the complex 6a appears as the light spot with low brightness because that second substance 11 is not positioned near the metal particle 2. Accordingly, the moving light spot can be automatically easily detected by image recognition, and false detection can be reduced. In addition, since even a fluorescent particle with a smaller particle size can also be recognized as the light spot, the fluorescent particle with the smaller particle size can be used in practice. As a result, it is possible to increase a reaction speed and to speed up the detection.

In the metal particle 2, the inner core 2a made of the magnetic material with paramagnetism or ferromagnetism is covered with the outer shell 2b made of the nonmagnetic metal material. Accordingly, the metal particles 2 can be suppressed from agglomerating together due to residual magnetization of the magnetic material. As a result, it is possible to suppress variations in brightness and moving speed of the light spot among the two-dimensional images and to increase the detection accuracy.

Since the surface of the metal particle 2 is covered with the metal material that produces the localized surface plasmon resonance, a variation in enhancement degree in a circumferential direction of the metal particle 2 can be suppressed in comparison with the case in which part of the surface of the metal particle 2 is made of the metal material.

Gold, silver, aluminum, or an alloy containing any of those metals as a main component can be used to form the outer shell, and the localized surface plasmon resonance can be effectively produced by the metal particle 2. Moreover, when the outer shell 2b is made of gold, coatings with various functions can be optionally easily formed on the surface of the metal particle 2. For example, in the case of forming a nonspecific adsorption preventive coating on the outer shell 2b, nonspecific adsorption that the second substance 11 labeled with the fluorescent particle 10 is adsorbed onto the surface of the metal particle 2 can be reduced, and the incidences of false-positive and false-negative detection results can be reduced.

### Second Embodiment

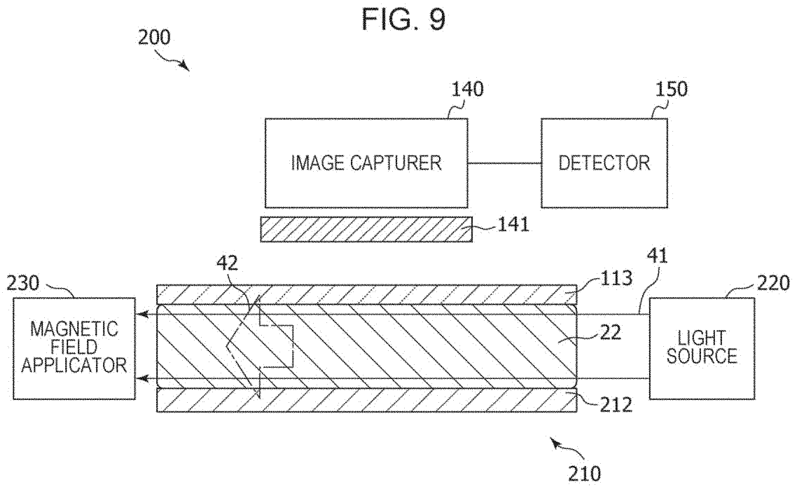

A second embodiment will be described next. The second embodiment is different from the above-described first embodiment in that the near field is not utilized. A detection device 200 according to the second embodiment will be described below with reference to FIG. 9 primarily about different points from the above-described first embodiment.

### Configuration of Detection Device

FIG. 9 illustrates a configuration of the detection device 200 according to the second embodiment. As illustrated in FIG. 9, the detection device 200 includes a sample container 210, a light source 220, a magnetic field applicator 230, the image capturer 140, the long-pass filter 141, and the detector 150.

As in the first embodiment, the sample container 210 contains the mixed solution 22 containing the complex 6a, the second substance 11 labeled with the fluorescent particle 10, and the first substance 3 immobilized to the metal particle 2. More specifically, the sample container 210 includes a substrate 212 and the cover glass 113. In this embodiment, the substrate 212 may not need to form the near field. Accordingly, the sample container 210 may not need to include the prism 111.

The light source 220 emits excitation light 41 with a predetermined wavelength. The predetermined wavelength may be set to the same wavelength as that of the excitation light 21 in the first embodiment. In the second embodiment, the light source 220 does not need to form the near field induced by the excitation light 41 and directly illuminates the mixed solution 22 with the excitation light 41. More specifically, the light source 220 emits the excitation light 41 to a space between the substrate 212 and the cover glass 113 in a direction parallel to the substrate 212 and the cover glass 113. With the excitation light 41 emitted in such a manner, the mixed solution 22 can be entirely illuminated with the excitation light 41, and the excitation light 41 can be suppressed from directly entering the image capturer 140.

Like the second magnetic field applicator 132 in the first embodiment, the magnetic field applicator 230 applies a magnetic field 42 that has a component in the horizontal direction (namely, in the direction parallel to the surface of the substrate 212) but does not have a component in any other direction. Thus, the magnetic field 42 has the horizontal component without having the component in any other direction. The first substance 3 immobilized to the metal particle 2 and the complex 6a in the mixed solution 22 are both moved in the horizontal direction by the magnetic field 42.

### Operation of Detection Device

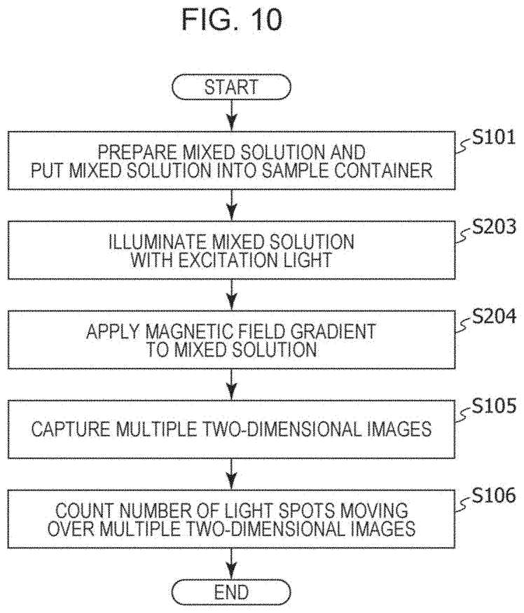

Operation of the detection device 200 according to the second embodiment is now described with reference to FIG. 10. FIG. 10 is a flowchart illustrating processing executed by the detection device 200 according to the second embodiment.

After the mixed solution 22 has been put into the sample container 210 in step S101, the light source 220 illuminates the mixed solution 22 with the excitation light 41 (S203). Then, the magnetic field applicator 230 applies the magnetic field 42 (S204). The magnetic field 42 is applied during a period in which the illumination with the excitation light 41 is continued.

Thereafter, step S105 and step S106 are executed as in the first embodiment.

### Advantageous Effects, Etc.

Also in this embodiment, as described above, the light spot of which position is changed over the two-dimensional images represents the fluorescence emitted from the complex 6a. Hence the detector 150 can detect the target substance 1 in accordance with the light spot of which position is changed over the two-dimensional images. On that occasion, as in the first embodiment, the first substance 3 not bound to the target substance 1 emits no fluorescence because of being not associated with the fluorescent particle 10. Furthermore, the fluorescence emitted from the second substance 11 not bound to the target substance 1 is not enhanced by the localized surface plasmon resonance. It is hence possible to increase a difference in brightness between the light spot corresponding to the complex 6a and the light spots corresponding to other substances, to facilitate the automatic image recognition of the moving light spot, and to reduce the false detection.

Moreover, since the fluorescence emitted from the complex 6a is enhanced by the localized surface plasmon resonance, even a fluorescent particle with a smaller particle size can also be recognized as the light spot, whereby the fluorescent particle with the smaller particle size can be utilized in practice. In addition, the application of the first magnetic field 23 by the first magnetic field applicator 131 in the first embodiment is no longer required in the second embodiment. As a result, the detection device 200 according to the second embodiment is effective in speeding up the detection.

### Example 1

### Simulation Model and Simulation Results

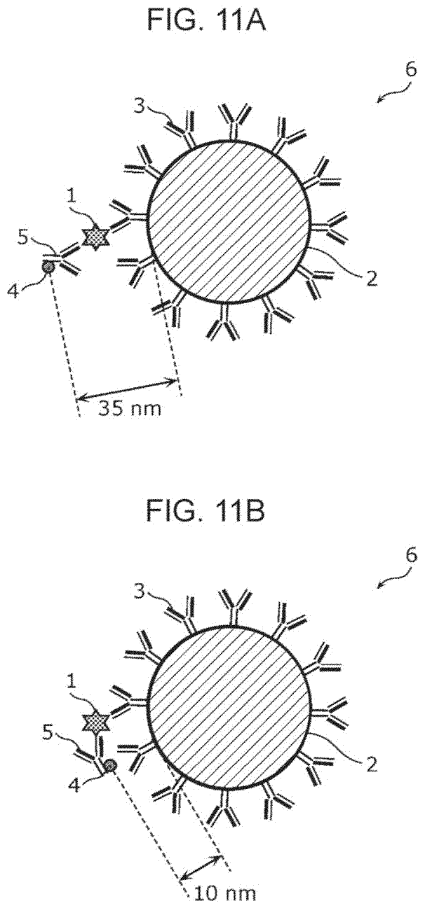

A simulation for the enhancement degree due to the localized surface plasmon resonance produced by the metal particle 2 will be described below as EXAMPLE 1 with reference to FIGS. 11A, 11B and 12 to 14.

In this EXAMPLE, serum albumin with a size of about 10 nm was used as the target substance 1. A core-shell type particle including the inner core 2a having a diameter of 13.6 nm and made of iron oxide ferrite and the outer shell 2b made of gold was used as the metal particle 2. A diameter of the metal particle 2 was 50 nm. An organic cyanine-based fluorescent molecule of greater than or equal to 1 nm, namely Cyanine 3 (Cy3, molecular weight: 714, excitation wavelength: (512); 550, fluorescence wavelength: 570; (615), and quantum yield QY: 0.15), was used as the fluorescent material 4. A monoclonal IgG antibody with a size of about 15 nm was used as each of the first substance 3 and the second substance 5.

When the metal particle 2, the fluorescent material 4, and the first and second substances 3 and 5 form the complex 6, a distance between the surface of the metal particle 2 and the fluorescent material 4 is different depending on a position (binding site) at which the second substance 5 is bound to the target substance 1. FIGS. 11A and 11B each illustrate a positional relationship between the metal particle 2 and the fluorescent material 4 in EXAMPLE 1. FIG. 11A represents a maximum value (about 35 nm) of the distance between the surface of the metal particle 2 and the fluorescent material 4 in EXAMPLE 1. On the other hand, FIG. 11B represents a minimum value (about 10 nm) of the distance between the surface of the metal particle 2 and the fluorescent material 4 in EXAMPLE 1.

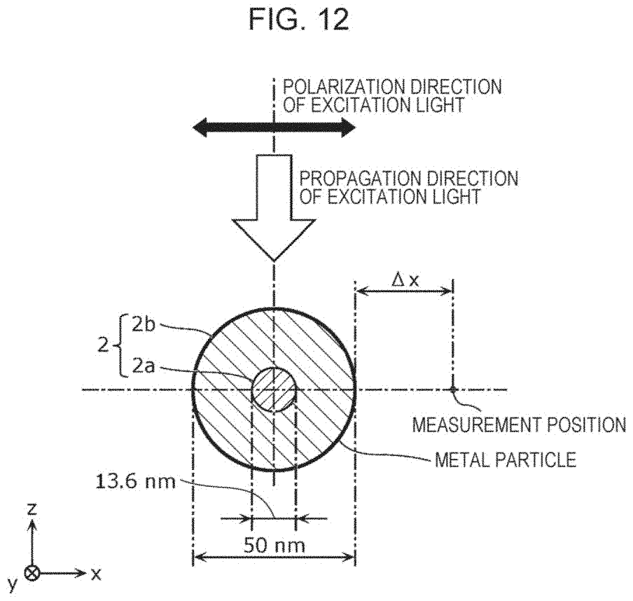

Here, the intensity of an electric field around the metal particle 2 was simulated by the FDTD (finite-difference time-domain) method. FIG. 12 is an explanatory view of a simulation model in EXAMPLES 1 and 2. In this EXAMPLE, as illustrated in FIG. 12, the core-shell type metal particle 2 having the diameter of 50 nm and being present in water was illuminated with a plane wave propagating in a negative direction of a z-axis. The plane wave was linearly polarized along the z-axis, and the intensity of an electric field of the plane wave was 1 [V/m]. In accordance with such a simulation model, the intensity of the electric field was calculated at a measurement position that was spaced Δx from the surface of the metal particle 2.

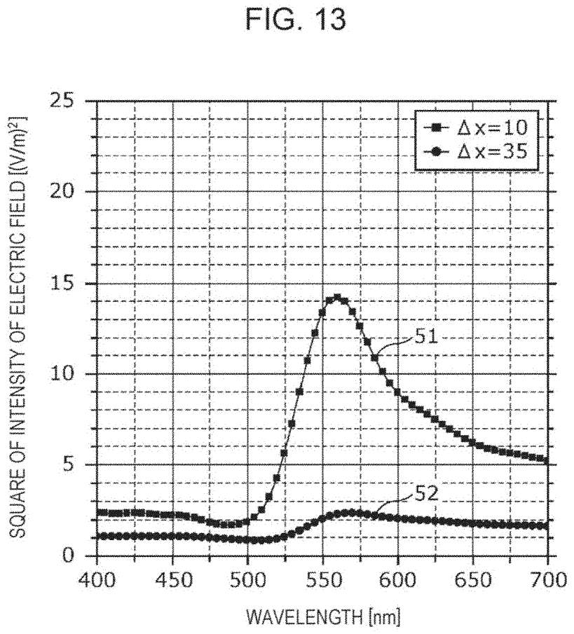

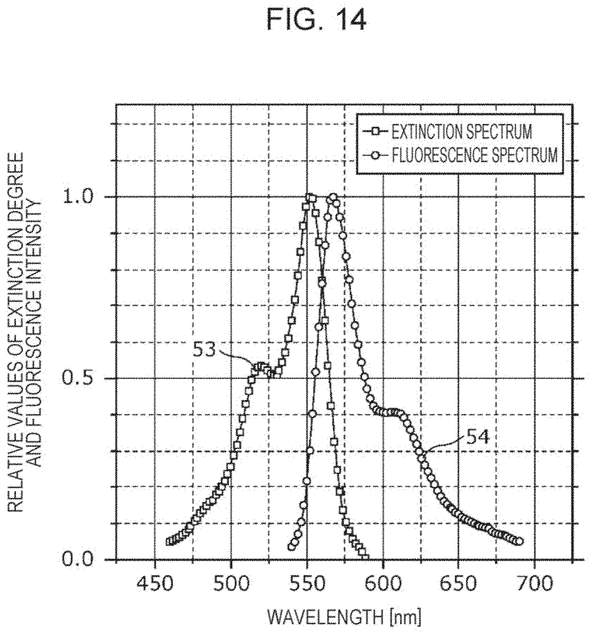

FIG. 13 is a graph depicting wavelength dependency of the intensity of the electric field near the metal particle in EXAMPLE 1. FIG. 13 represents the simulation results. In FIG. 13, a horizontal axis indicates the wavelength of the excitation light, and a vertical axis indicates the square ((V/m)²) of the intensity of the electric field. A data point 51 represents a square value of the intensity of the electric field at the measurement position that was spaced 10 nm away from the surface of the metal particle 2 (namely, Δx=10 nm in FIG. 12). A data point 52 represents a square value of the intensity of the electric field at the measurement position that was spaced 35 nm away from the surface of the metal particle 2 (namely, Δx=35 nm in FIG. 12). The square of the intensity of the electric field corresponds to the enhancement degree. As is apparent from FIG. 13, the localized surface plasmon resonance occurs in a wavelength range of about 500 to 600 nm.

An extinction spectrum and a fluorescence spectrum of Cy3 used as the fluorescent material 4 is described next. FIG. 14 is a graph depicting the extinction spectrum and the fluorescence spectrum of the fluorescent material 4 in EXAMPLE 1. In FIG. 14, a horizontal axis indicates a wavelength, and a vertical axis indicates a relative value of each of an extinction degree and fluorescence intensity. Here, the relative value is given as a value resulting from normalizing a value range of each of the extinction spectrum and the fluorescence spectrum into a range from 0 to 1, A data point 53 represents the extinction spectrum, and a data point 54 represents the fluorescence spectrum.

As seen from FIGS. 13 and 14, the wavelength range of the localized surface plasmon resonance produced by the metal particle 2 with the diameter of 50 nm, a wavelength range for exciting Cy3, and a wavelength range of the fluorescence emitted from Cy3 overlap with one another. Accordingly, the excitation light incident on Cy3 and the fluorescence emitted from Cy3 are enhanced by the action of the localized surface plasmon resonance.

### Enhancement Degrees for Excitation Light and Fluorescence

The enhancement degree for the excitation light of a wavelength 532 nm is now described with reference to FIG. 13. From FIG. 13, the enhancement degrees for the excitation light at the positions of the fluorescent material 4, denoted in FIGS. 11A and 11B, are given as follows.

EF(532, 35)=1.3

EF(532, 10)=8.0

Here, EF(λ, Δx) represents the enhancement degree (the square of the intensity of the electric field) for light of a wavelength λ at the position given by Δx. Thus, EF(532, 35) represents the enhancement degree for the excitation light at the position (Δx=35) of the fluorescent material 4 in FIG. 11A, and EF(532, 10) represents the enhancement degree for the excitation light at the position (Δx=10) of the fluorescent material 4 in FIG. 118.

As understood from the above discussion, at the positions of the fluorescent material 4 denoted in FIGS. 11A and 11B, the excitation light is enhanced 1.3 times and 8 times, respectively, in comparison with the case in which the metal particle 2 is not present. Such excitation enhancement is resulted from the fact that the excitation light is collected to the surroundings of the metal particle 2 due to the localized surface plasmon resonance.

The enhancement degree for the fluorescence emitted from the fluorescent material 4 upon being illuminated with the above-mentioned excitation light is described next. Cy3 excited by the excitation light of the wavelength 532 nm emits the fluorescence exhibiting a spectroscopic spectrum with a peak wavelength of 570 nm (see the fluorescence spectrum in FIG. 14). From FIG. 13, the enhancement degrees for the fluorescence of the wavelength 570 nm at the different positions of the fluorescent material 4 are given as follows.

EF(570, 35)=2.4

EF(570, 10)=13

As understood from the above discussion, at the positions of the fluorescent material 4 denoted in FIGS. 11A and 11B, the fluorescence emitted from Cy3 is enhanced 2.4 times and 13 times, respectively, in comparison with the case in which the metal particle 2 is not present. Such emission enhancement is resulted from the fact that the quantum yield of the fluorescent material 4 around the metal particle 2 is increased due to the localized surface plasmon resonance.

The quantum yield of Cy3 is 0.15 usually (namely, in the case in which there are no metal particles 2 around Cy3). Because a maximum value of the quantum yield is 1, a maximum value of the emission enhancement for Cy3 is given by 1/0.15≈6.7. Thus, a maximum value of the enhancement degree EF(570, Δx) is held down to 6.7, and EF(570, 10) is replaced with the following value.

EF(570, 10)=6.7

### Enhancement Degree for Surface Enhanced Fluorescence

Because the surface enhanced fluorescence is generated due to both the excitation enhancement and the emission enhancement, the enhancement degree for the surface enhanced fluorescence is calculated by the product of the enhancement degree of the excitation enhancement and the enhancement degree of the emission enhancement. Accordingly, the enhancement degree for the surface enhanced fluorescence emitted from Cy3 is calculated as follows.

SEF(35)=EF(532,35)×EF(570,35)=1.3×2.4=3.1

SEF(10)=EF(532,10)×EF(570,10)=8×6.754

where SEF(Δx) represents the enhancement degree for the surface enhanced fluorescence at the position given by Δx.

In this EXAMPLE, as described above, the fluorescence emitted from the fluorescent material 4 (Cy3) contained in the complex 6 was enhanced 3.1 to 54 times in comparison with the fluorescence emitted from the fluorescent material 4 (Cy3) that was not contained in the complex 6.

### Example 2

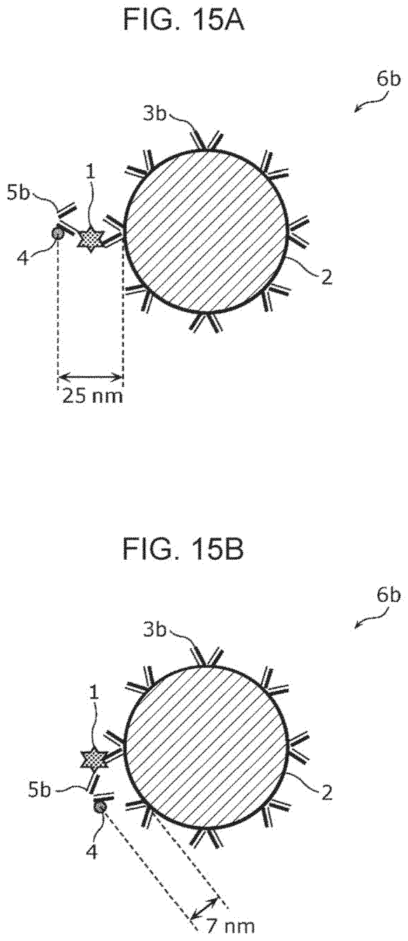

Simulation results of the enhancement degree due to the localized surface plasmon resonance produced by the metal particle 2 in the case of using an antibody smaller than that used in EXAMPLE 1 will be described below as EXAMPLE 2. EXAMPLE 2 is described mainly about different points from EXAMPLE 1 with reference to FIGS. 15A, 15B, 16 and 17.

### Simulation Model and Simulation Results

In this EXAMPLE 2, a fragment antibody F(ab′)2 smaller than the IgG antibody used in EXAMPLE 1 was used as each of a first substance 3b and a second substance 5b. The fragment antibody F(ab′)2 was an antibody obtained by fragmenting the IgG antibody and was prepared by decomposing the IgG antibody with pepsin that is a proteolytic enzyme. F(ab′)2 included a hinge site at the N-terminal end of the IgG antibody, and two antibody binding portions were bounded to each other at the hinge site. A size of F(ab′)2 was about a half that of IgG antibody and was about 7 nm.

When the complex 6b is formed using F(ab′)2, a distance between the surface of the metal particle 2 and the fluorescent material 4 is different depending on a position (binding site) at which the second substance 5b is bound to the target substance 1. FIGS. 15A and 153 each illustrate a positional relationship between the metal particle 2 and the fluorescent material 4 in EXAMPLE 2. FIG. 15A represents a maximum value (about 25 nm) of the distance between the surface of the metal particle 2 and the fluorescent material 4. On the other hand, FIG. 15B represents a minimum value (about 7 nm) of the distance between the surface of the metal particle 2 and the fluorescent material 4.

Here, as in EXAMPLE 1, the intensity of an electric field around the metal particle 2 was simulated by the FDTD method. A simulation model was similar to that used in EXAMPLE 1, Hence the drawing and the description of the simulation model are omitted.

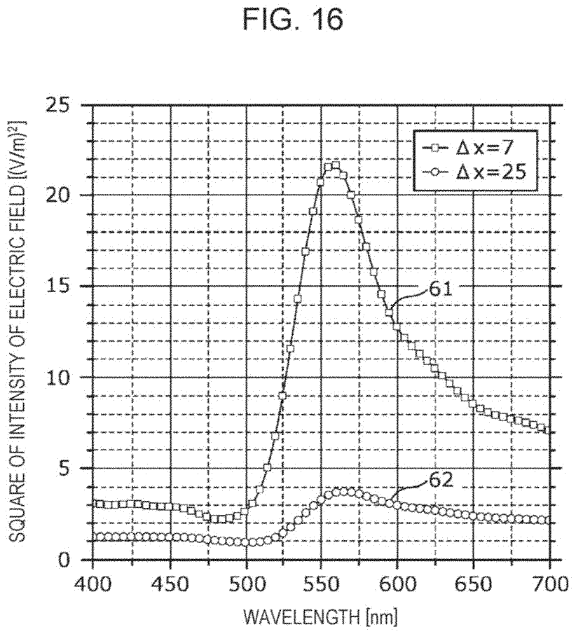

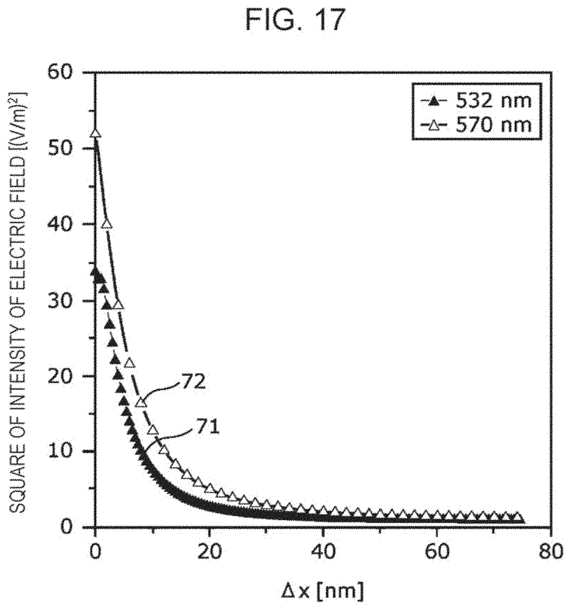

FIG. 16 is a graph depicting wavelength dependency of the intensity of the electric field near the metal particle in EXAMPLE 2. FIG. 16 represents the simulation results. In FIG. 16, a horizontal axis indicates the wavelength of the excitation light, and a vertical axis indicates the square ((V/m)²) of the intensity of the electric field. A data point 61 represents a square value of the intensity of the electric field at the measurement position that was spaced 7 nm away from the surface of the metal particle 2 (namely, Δx=7 nm in FIG. 12). A data point 62 represents a square value of the intensity of the electric field at the measurement position that was spaced 25 nm away from the surface of the metal particle 2 (namely, Δx=25 nm in FIG. 12). As seen from FIGS. 14 and 16, the wavelength range of the localized surface plasmon resonance, the wavelength range for exciting Cy3, and the wavelength range of the fluorescence emitted from Cy3 overlap with one another as in EXAMPLE 1. Accordingly, the excitation light incident on Cy3 and the fluorescence emitted from Cy3 are enhanced by the action of the localized surface plasmon resonance.

### Enhancement Degrees for Excitation Light and Fluorescence

The enhancement degree for the excitation light of the wavelength 532 nm is now described with reference to FIG. 16. From FIG. 16, the enhancement degrees for the excitation light at the positions of the fluorescent material 4, denoted in FIGS. 15A and 15B, are given as follows.

EF(532, 25)=2.0

EF(532, 7)=13

As understood from the above discussion, at the positions of the fluorescent material 4 denoted in FIGS. 15A and 15B, the excitation light emitted from Cy3 is enhanced 2 times and 13 times, respectively, in comparison with the case in which the metal particle 2 is not present.

The enhancement degree for the fluorescence emitted from the fluorescent material 4 upon being illuminated with the above-mentioned excitation light is described next. Cy3 excited by the excitation light of the wavelength 532 nm emits the fluorescence exhibiting a spectroscopic spectrum with a peak wavelength of 570 nm (see the fluorescence spectrum in FIG. 14). From FIG. 16, the enhancement degrees for the fluorescence of the wavelength 570 nm at the different positions of the fluorescent material 4 are given as follows.

EF(570, 25)=3.7

EF(570, 7)=20

As understood from the above discussion, at the positions of the fluorescent material 4 denoted in FIGS. 15A and 15B, the fluorescence emitted from Cy3 is enhanced 3.7 times and 20 times, respectively, in comparison with the case in which the metal particle 2 is not present, Such emission enhancement is resulted from the fact that the quantum yield of the fluorescent material 4 around the metal particle 2 is increased due to the localized surface plasmon resonance.

As in EXAMPLE 1, because of limitation on a maximum value (=1) of the quantum yield of Cy3, a maximum value of the emission enhancement for Cy3 is given by 1/0.15≈6.7. Thus, the enhancement degree EF(570, 7) is held down to 6.7, and EF(570, 7) is replaced with the following value as in EXAMPLE 1

EF(570, 7)=6.7

### Enhancement Degree for Surface Enhanced Fluorescence

Because the surface enhanced fluorescence is generated due to both the excitation enhancement and the emission enhancement, the enhancement degree for the surface enhanced fluorescence is calculated by the product of the enhancement degree of the excitation enhancement and the enhancement degree of the emission enhancement. Accordingly, the enhancement degree SEF(Δx) for the surface enhanced fluorescence emitted from Cy3 is calculated as follows.

SEF(25)=EF(532,25)×EF(570,25)=2.0×3.7=7.4

SEF(7)=EF(532,7)×EF(570,7)=13×6.787

In this EXAMPLE, as described above, the fluorescence emitted from the fluorescent material 4 (Cy3) contained in the complex 6b was enhanced 7.4 to 87 times in comparison with the fluorescence emitted from the fluorescent material 4 (Cy3) that was not contained in the complex 6b.

### Distance Dependency of Enhancement Degree

As is apparent from EXAMPLE 1 and EXAMPLE 2, the enhancement degrees for the excitation light and the fluorescence change depending on the distance from the metal particle 2. From that point of view, FDTD simulation results for distance dependency of the enhancement degrees for the excitation light and the fluorescence will be described below with reference to FIG. 17.

FIG. 17 is a graph depicting the distance dependency of the intensity of the electric field near the metal particle 2 in EXAMPLES 1 and 2, In FIG. 17, a horizontal axis indicates the distance Δx from the metal particle 2, and a vertical axis indicates the enhancement degree. A data point 71 represents the enhancement degree for the excitation light of the wavelength 532 nm. A data point 72 represents the enhancement degree for the fluorescence of the wavelength 570 nm.

As seen from FIG. 17, since the enhancement degree increases as Δx decreases, the enhancement degree for the surface enhanced fluorescence can be increased by selecting the size and the binding site of the antibody such that Δx decreases. However, when the distance Δx between the fluorescent material 4 and the metal particle 2 is reduced below 5 nm, there occurs a fluorescence quenching phenomenon due to direct transfer of energy from the fluorescent material 4 to the metal particle 2, Therefore, Δx may be set not to be shorter than 5 nm (see Anger. P.; Bharadwaj, P.; Novotny, L. PhysRevLett. 96.113002 (2006)).

EXAMPLE 1 and EXAMPLE 2 described above are practically implemented because a highly practical and widespread DPSS (diode pumped solid state) laser can be used as the light source 120.

Furthermore, according to EXAMPLE 2, since the smaller antibody than in EXAMPLE 1 is utilized, a higher enhancement degree for the surface enhanced fluorescence can be obtained and the target substance 1 can be detected with higher sensitivity than in EXAMPLE 1.

The advantageous effects described in connection with the foregoing embodiments can be similarly obtained in both the cases of using the antibodies in EXAMPLE 1 and EXAMPLE 2. However, when, for example, a polystyrene particle larger than the fluorescent material 4 is used as the fluorescent particle 10, the advantageous effects become more restrictive than those obtained with the simulation results in EXAMPLE 1 and EXAMPLE 2 for the reason that the distance from the surface of the metal particle 2 cannot be consistently approximated by using Δx.

### MODIFICATIONS

The detection device according to one or more aspects of the present disclosure has been described in connection with the embodiments, but the present disclosure is not limited to those embodiments, Other embodiments that are obtained by variously modifying those embodiments in accordance with the ideas conceivable by those skilled in the art and that are constituted by combining the components in the different embodiments with each other may also fall within the scope of the one or more aspects of the present disclosure insofar as not departing from the gist of the present disclosure.

For example, in the above-described embodiments, the first substance and the second substance are each not limited to the IgG antibody and the fragment antibody F(ab′)2 used in EXAMPLES 1 and 2. In another example, a different type of fragment antibody, such as Fab′, Fab, Fv, or scFv having a single binding portion, may also be used instead of F(ab′)2. In still another example, a fragment of VHH (variable domain of heavy chain of heavy chain antibody) (nanobody) obtained from a Camelidae animal (such as llama or alpaca) may also be used. Furthermore, the first substance and the second substance are not limited to antibodies insofar as they specifically bind to the target substance, and may be each an aptamer such as a nucleic acid molecule or peptide.

The present disclosure is applied to a sensor device for detecting the target substance in a simple manner at a high speed with high accuracy.

What is claimed is:

1. A detection method comprising:
forming a complex by binding, to a target substance, a first substance immobilized to a metal particle with magnetism and a second substance labeled with a fluorescent material;
moving the complex by applying a magnetic field;
illuminating the complex during movement with excitation light of a predetermined wavelength, the excitation light causing the fluorescent material to emit fluorescence, the fluorescence being enhanced by localized surface plasmon resonance that is produced by the metal particle;
capturing the enhanced fluorescence over time and obtaining two-dimensional images; and
detecting the target substance in accordance with a light spot included in each of the two-dimensional images,
the metal particle including an inner core made of a magnetic material and an outer shell covering the inner core, the outer shell being made of a nonmagnetic metal material that produces the localized surface plasmon resonance.

2. The detection method according to claim 1, wherein the illuminating the complex with the excitation light includes illuminating a substrate capable of forming a near field induced by the excitation light with the excitation light, thereby the complex positioned near a surface of the substrate being illuminated with the near field induced by the excitation light.

3. The detection method according to claim 2, wherein the applying the magnetic field includes:
applying a first magnetic field, thereby the complex being attracted toward the surface of the substrate, and
applying a second magnetic field, thereby the complex attracted to the surface of the substrate moving along the surface of the substrate.

4. The detection method according to claim 1, wherein the magnetic material contains a paramagnetic substance, and the nonmagnetic metal material contains a diamagnetic substance.

5. The detection method according to claim 1, wherein the nonmagnetic metal material is gold, silver, aluminum, or an alloy containing, as a main component, one of gold, silver, and aluminum.

6. A detection device comprising:
a sample container containing a sample including a complex that is formed by binding, to a target substance, a first substance immobilized to a metal particle with magnetism and a second substance labeled with a fluorescent material;
a magnetic field applicator applying a magnetic field to the sample contained in the sample container, thereby the magnetic field moving the complex;
a light source illuminating the sample contained in the sample container with excitation light of a predetermined wavelength, the excitation light causing the fluorescent material to emit fluorescence, the fluorescence being enhanced by localized surface plasmon resonance that is produced by the metal particle;
an image captures capturing the enhanced fluorescence over time and obtaining two-dimensional images; and
a detector detecting the target substance in accordance with a light spot included in each of the two-dimensional images,
the metal particle including an inner core made of a magnetic material and an outer shell covering the inner core, the outer shell being made of a nonmagnetic metal material that produces the localized surface plasmon resonance.

7. The detection device according to claim 6, wherein the sample container includes a substrate capable of forming a near field upon being illuminated with the excitation light, and
the light source illuminates the substrate with the excitation light, thereby the complex positioned near a surface of the substrate being illuminated with the near field induced by the excitation light.

8. The detection device according to claim 7, wherein the magnetic field applicator includes:
a first magnetic field applicator applying a first magnetic field to the sample and attracting the complex toward the surface of the substrate, and
a second magnetic field applicator applying a second magnetic field to the sample and moving the complex along the surface of the substrate.

9. The detection device according to claim 6, wherein the magnetic material contains a paramagnetic substance, and the nonmagnetic metal material contains a diamagnetic substance.

10. The detection device according to claim 6, wherein the nonmagnetic metal material is gold, silver, aluminum, or an alloy containing, as a main component, one of gold, silver, and aluminum.
